(19) United States
(12) Patent Application Publication
(10) Pub. No.: US 2010/0247672 A1
(43) Pub. Date: Sep. 30, 2010

(54) POLYURETHANE/BONE COMPOSITIONS AND METHODS

(76) Inventors: Scott A. Guelcher, Franklin, TN (US); Jerald Dumas, Nashville, TN (US)

Correspondence Address: STITES & HARBISON PLLC, 401 COMMERCE STREET, SUITE 800, NASHVILLE, TN, 37219, US

(21) Appl. No.: 12/676,700

(22) PCT Filed: Sep. 5, 2008
(86) PCT No.: PCT/US08/75498

§ 371 Date: Jun. 15, 2010

Related U.S. Application Data
(60) Provisional application No. 60/970,205, filed on Sep. 5, 2007.

(51) Int. Cl. A61K 35/32; A61F 2/28

(52) U.S. Cl. 424/549; 623/23.58; 523/115

(57) ABSTRACT

A flowable, injectable composite that comprises mineralized allograft bone; and at least one degradable polyurethane that has a quasi-prepolymer and a resin mix, the resin mix having a polyester polyol and a catalyst; wherein the composite has a compression strength of greater than about 10 MPa and a modulus of greater than about 1 GPa.

### PRIORITY INFORMATION

This application claims benefit to U.S. Patent Application No. 60/970,205, the contents of which are incorporated herein by reference in their entirety.

### GOVERNMENT SUPPORT

This invention was made with support from Department of Defense grant number DOD-2110-PO5-694362. The United States Government has rights to this invention.

### BACKGROUND OF THE INVENTION

The need for orthopedic care to address vertebral fractures/defects is becoming an increasing demand due to the growing elderly population in the U.S. Like most surgical procedures, vertebral surgeries can require extensive post-operative care. In order to reduce the recovery time, several minimum invasive surgical techniques have been developed to repair such defects. However, an injectable technology that is osteoconductive, load bearing and resorbable currently does not currently exist.

### SUMMARY OF THE INVENTION

Aspects of the present invention relate to weight-bearing composites of mineralized bone and reactive, two-component biodegradable polyurethanes. These materials have been shown in previous studies to degrade and remodel in vivo (Knaack 2003, boyce 2005). The materials are prepared by contacting a polyisocyanate quasi-prepolymer, a resin mix incorporating a polyester polyol (optionally also a polyether polyol), and a catalyst. The mineralized bone can be added to either the polyisocyanate or resin mix component or both prior to mixing of the polyisocyanate quasi-prepolymer and resin mix.

One embodiment of the present invention is a bone composite material that comprises lysine diisocyanate (LDI), polycaprolactone, and mineralized bone powder (MBP). In embodiments of the invention, the polycaprolactone may be of a molecular weight of about 300 (PCL 300); the bone powder is about 100-500 microns. With respect to the material, the bone may be present in an amount of, for example, about 75 wt %, about 85 wt %, about 60 to about 83 wt %, about 70 to about 80 wt %. In embodiments, the bone powder is surface activated.

Preferably the composite material is flowable. Thus, the material may be injected at a would site, allowing for a less invasive application.

In another embodiment of the present invention is provided a composite that comprises mineralized allograft bone; and at least one degradable polyurethane that has a quasi-prepolymer and a resin mix, the resin mix having a polyester polyol and a catalyst; wherein the composite has a compression strength of greater than about 10 MPa and a modulus of greater than about 1 GPa. In aspects of this embodiment, the bone may be in powder form when integrated into the composite and, in aspects of the invention, present in a range of from about 1-90 wt %, or about 50-85 wt %, or about 70-85 wt %.

Another embodiment of the present invention is a method of treating a wound that comprises providing mixture of mineralized allograft bone; and at least one degradable polyurethane that has a quasi-prepolymer and a resin mix, the resin mix having a polyester polyol and a catalyst; and injecting the mixture into a wound site. In aspects of this embodiment, the wound site is a bone.

Another embodiment of the present invention is a method of treating a wound that comprises providing mixture of mineralized allograft bone; and at least one degradable polyurethane that has a quasi-prepolymer and a resin mix, the resin mix having a polyester polyol and a catalyst; compression molding the mixture; and contacting the molding to the wound site. In aspects of this embodiment, the wound site is a bone.

Yet another embodiment of the present invention is a method for preparing biodegradable polyurethanes comprising: contacting a flowable quasi-prepolymer comprising free aliphatic polyisocyanate compounds with a polyester polyol hardener having a functionality of at least two and mineralized bone powder to form a reactive liquid mixture.

In aspects of this embodiment, the quasi-prepolymer is formed by contacting a polyisocyanate component comprising at least one aliphatic polyisocyanate compound with a polyol component comprising at least one polyol compound to form an adduct of the polyisocyanate component and the polyol component wherein a sufficient excess of the polyisocyanate component is used to form the quasi-prepolymer. As examples, the polyester polyol may comprise poly(ethylene adipate), poly(ethylene glutarate), poly(ethylene azelate), poly(trimethylene glutarate), poly(pentamethylene glutarate), poly(diethylene glutarate), poly(diethylene adipate), poly(triethylene adipate), poly(1,2-propylene adipate), a mixture thereof, or a copolymer of at least two thereof. In other aspects of this embodiment, the polyester polyol may comprise polyesters prepared from at least one of ε-caprolactone, glycolide or DL-lactide.

Another embodiment of the present invention is a biodegradable polyurethane formed by contacting a flowable quasi-prepolymer comprising free aliphatic polyisocyanate compounds with a polyester polyol hardener having a functionality of at least two and mineralized bone powder to form a reactive liquid mixture.

Yet another embodiment of the present invention is a bone scaffold comprising a biodegradable polyurethane and bone powder as set forth herein.

### BRIEF DESCRIPTION OF THE DRAWINGS

FIG. 1 is a graph showing DMA temperature sweep of MBP/PEUR composite, which shows a glass transition temperature of 66° C.

FIG. 2 shows MC3T3 cells seeded on the composite surface, (A) Day 1 Hoescht blue (HB) staining in osteogenic media (OS+), (B) Day 4 HB staining (OS+), (C) OS+ interface between composite and culture plate, (D) OS− interface.

### DESCRIPTION OF THE INVENTION

The present invention provides compositions and methods for allograft bone/poly(ester urethane) composite devices intended for use in fracture fixation devices. An advantage of the composites of the present invention is that over time the bone remodels and the polymer degrades, whereas metal fracture fixation devices do not remodel. Through the reactive liquid molding process, the binding strength between the polymer and bone phases is improved relative to composites prepared from thermoplastic polymers.

Embodiments of the present invention include a two-component reactive liquid approach in which mineralized bone/poly(ester urethane) composites are mixed at ambient temperature, thus rendering them useful for injectable applications. Furthermore, by enhancing the reactivity of the surface of the bone particles, the interfacial binding, and therefore the mechanical properties, can be improved. Examples of composites of the present incorporate about 75 wt % bone over a range of indices (index=100×NCO:OH equivalent ratio) and over a range of polyester polyol molecular weights. Other embodiments include up to about 85 wt % bone fractions. Other embodiments include from about 60 to about 83 wt % bone fractions. Other embodiments include about 70-80 wt %.

As indicated herein, the composites of the present invention are useful biomaterials for bone tissue engineering. Catalyst levels can be manipulated to adjust process time with the option of using either the Coscat 83 or Tegoamin 33. By varying the molecular weight of the polyol, modulus of the injected MBP/PEUR composites of the present invention can tuned based on its specific application. Similarly, the results from surface activation of the MBP suggest that activation does enhance the mechanical properties of the by improving interfacial binding between the MBP and PEUR phase. MC3T3 cells adhered to the composites and displayed no evidence of being cytotoxic. Also, cell proliferation occurred on the composite surface between days 1 and 4 regardless of the medial type, further indicating that the MBP/PEUR composites are biocompatible.

More specifically, an example of a polymer that may be used in connection with the instant invention is one that is disclosed in WO2007123536, the contents of which are incorporated herein by reference.

Thus, in the quasi-prepolymer process of the present invention, the quasi-prepolymer can be prepared by contacting a polyol component including at least one polyol compound with an excess (typically a large excess) of a polyisocyanate component. The resulting quasi-prepolymer intermediate includes an adduct of polyisocyanate and polyol solubilized in an excess of polyisocyanate. The quasi-prepolymer can also be formed by using an approximately stoichiometric amount of polyisocyanate component in forming a prepolymer and subsequently adding additional polyisocyanate component. The quasi-prepolymer therefore exhibits both low viscosity, which facilitates processing, and improved miscibility as a result of the polyisocyanate-polyol adduct. Poly(ester urethane) (PEUR) networks can, for example, then be prepared by reactive liquid molding, wherein the quasi-prepolymer is contacted with a polyester polyol to form a reactive liquid mixture which is then cast into a mold and cured.

Suitable polyisocyanate compounds or multi-isocyanate compounds for use in the present invention include aliphatic polyisocyanate compounds. Suitable aliphatic polyisocyanate compounds include, but are not limited to, lysine diisocyanate, an alkyl ester of lysine diisocyanate (for example, the methyl ester or the ethyl ester), lysine triisocyanate, hexamethylene diisocyanate, isophorone diisocyanate (IPDI), 4,4′-dicyclohexylmethane diisocyanate (H₁₂MDI), cyclohexyl diisocyanate, 2,2,4-(2,2,4)-trimethylhexamethylene diisocyanate (TMDI), dimers prepared form aliphatic polyisocyanates, trimers prepared from aliphatic polyisocyanates and/or mixtures thereof. In general, the polyisocyanate used in the present invention preferably includes approximately 10 to 55% NCO by weight (wt % NCO=100*(42/Mw)). More preferably, the polyisocyanates include approximately 15 to 50% NCO.

Suitable polyol compounds for use in the polyol component in preparation of the quasi-prepolymers of the present invention include, but are not limited to, starter compounds having a hydroxy functionality of at least 3 and/or polyester polyols. Preferably, such starter compounds have a molecular weight of no more than 300 g/mol. Starter compounds suitable for use in the present invention include, but are not limited to, at least one of glycerol, pentaerythritol, dipentaerythritol, tripentaerythritol, 1,2,4-butanetriol, trimethylolpropane, 1,2,3-trihydroxyhexane, myo-inositol, ascorbic acid, a saccharide, or sugar alcohols (for example, mannitol, xylitol, sorbitol etc.). The quasi-prepolymer can also include other compounds having multiple reactive hydrogen functional groups (for example, hydroxy groups, primary amine groups and/or secondary amine groups) to react with the isocyanate functionality of the polyisocyanate compound(s). Hydroxy functional compounds are preferred.

Suitable polyester polyols for use in the present invention (in the polyol component used in synthesizing the quasi-prepolymer and/or in the polyester polyol added to the quasi-prepolymer in preparation of a reactive liquid mixture) include polyester polyols having an average hydroxy functionality greater than 2 and including hydrolysable polyester linkages. The polyester polyol can, for example, include polyalkylene glycol esters or polyesters prepared from cyclic esters. The polyester polyol can, for example, include poly(ethylene adipate), poly(ethylene glutarate), poly(ethylene azelate), poly(trimethylene glutarate), poly(pentamethylene glutarate), poly(diethylene glutarate), poly(diethylene adipate), poly(triethylene adipate), poly(1,2-propylene adipate), mixtures thereof, and/or copolymers thereof. The polyester polyol can also include, polyesters prepared from ε-caprolactone, glycolide, DL-lactide, mixtures thereof, and/or copolymers thereof. The polyester polyol can also, for example, include polyesters prepared from castor-oil.

In general, it is preferred that polyols and other compounds used in forming the quasi-prepolymer be miscible with the polyester polyol(s) used in forming the reactive liquid mixtures of the present invention. Moreover, it is desirable that the quasi-prepolymer be a flowable liquid at processing conditions. In general, the processing temperature is preferably no greater than 60° C. More preferably, the processing temperature is ambient temperature (25° C.)). Thus, polyols can be chosen to have a glass transition (Tg) temperature less than 60° C., less than 37° C. or even less than 25° C. Because the quasi-prepolymer is solubilized with excess polyisocyanate, however, compounds having glass transition temperatures higher than the processing temperature can be used. The molecular weight of the polyol(s) used in forming the quasi-prepolymer are preferably in the range of approximately 50 to 10,000 Da, more preferably in the range of approximately 50 to 3000 Da and, even more preferably, in the range of approximately 50 to 2000 Da. In general, the viscosity of the quasi-prepolymer is preferably matched to the viscosity of the polyester polyol (hardener) used to form the reactive liquid mixture. The viscosity of the quasi-prepolymers of the present invention is less than 80,000 cSt. In general, the viscosity of the quasi-prepolymer is preferably less than 10,000 cSt, more preferably less than 5000 cSt and, even more preferably, less than 3000 cSt.

The glass transition temperature of the polyester polyol used in forming the reactive liquid is preferably less than 60° C., less than 37° C. (approximately human body temperature) or even less than 25° C. In addition to affecting flowability at processing conditions, Tg can also affect degradation. In general, a Tg of greater than approximately 37° C. will result in slower degradation within the body, while a Tg below approximately 37° C. will result in faster degradation.

The molecular weight of the polyester polyol hardener used in forming the reactive liquid can, for example, be used to control the mechanical properties of the PEUR networks of the present invention. In that regard, using polyester polyols of higher molecular weight results in greater compliance or elasticity. The polyester polyol(s) used in forming the reactive liquids of the present preferably have a molecular weight less than approximately 20,000 Da. More preferably, the molecular weight is approximately in the range of 100 to 5000 Da. Even more preferably, the molecular weight is in the range of approximately 100 to 3000 Da.

A crosslinker can, for example, be added to the polyester polyol before contacting the quasi-prepolymer with the polyester polyol. Preferably, the crosslinker has a functionality of at least 3 and a molecular weight of no more than 300 g/mol. The crosslinker can, for example, include at least one of glycerol, pentaerythritol, dipentaerythritol, tripentaerythritol, 1,2,4-butanetriol, trimethylolpropane, 1,2,3-trihydroxyhexane, myoinositol, ascorbic acid, a saccharide, or a sugar alcohol (for example, (for example, mannitol, xylitol, sorbitol etc.).

In several representative examples of the present invention, poly(ε-caprolactone) triol (referred to herein as PCL) triol, having a glass transition temperature or Tg of −63° C., or poly(ε-caprolactone-co-glycolide-co-DL-lactide) triol (referred to herein as P6C3G1L), having a molecular weight of 300 Da and a Tg of −36° C., were reacted with lysine methyl ester diisocyanate (LDI) and lysine triisocyanate (LTI) via a two-step quasi-prepolymer process to produce biodegradable and biocompatible poly(ester urethane) networks. Amorphous polyester triol compositions with Tg less than ambient temperature (approximately 25° C.) were chosen to facilitate processing by reactive liquid molding. The composition of the polyester triols was varied as set forth in Table 1 to form four differenet prepolymers (designated QTPCL, QTP6C3G1L, QDPC and QDP6C3G1L as set forth in Table 1) to, for example, investigate the effects on PEUR degradation in vitro. Coscat 83 was used as a catalyst in the preparation of each quasi-prepolymer. In general, any conventional urethane catalyst can be used in the present invention. Preferred catalysts include catalysts exhibiting relatively low toxicity such as organobismuth compounds and tertiary amines. Organobismuth compounds are particularly preferred.

TABLE 1

| Prepolymer | QTPCL | QTP6C3G1L | QDPCL | QDP6C3G1L |
|---|---|---|---|---|
| Polyol | PCL | P6C3G1L | PCL | P6C3G1L |
| Polyisocyanate | LTI | LTI | LDI | LDI |
| Coscat 83 (ppm) | 466 | 466 | 400 | 908 |
| NCO:OH eq ratio | 7.10 | 7.10 | 4.04 | 4.04 |
| Polyisocyanate:polyol | 7.10 | 7.10 | 6.07 | 6.07 |
| molar ratio | | | | |
| % free NCO | 34.5% | 34.5% | 23.9% | 23.9% |

Further details and representative examples of the present invention are described in the following examples, which are presented to show embodiments of the present invention and are not to be construed as being limiting thereof.

### EXAMPLES

Materials and Methods. Bone/poly(ester urethane) (PEUR) composites are composed of lysine diisocyanate (LDI), polycaprolactone (molecular weight 300) (PCL 300), and mineralized bone powder (100-500 microns) (MBP). Coscat 83, an organobismuth catalyst, and Tegoamin 33, an amine catalyst, were both studied as potential catalysts. Polymers synthesized from LDI and polyester polyols have been reported to support cell attachment and biodegrade to non-cytotoxic degradation products. Surface activated bone, etched using an adapted procedure from Osteotech, Inc, was interchanged with the unmodified MBP. During the polyurethane reaction temperature was monitored using a temperature probe to monitor the heat of reaction. Samples were cast as 11 mm×11 mm cylinders for compression testing, which was conducted on a MTS with a 13 kN load cell. Dynamic mechanical properties were measured by dynamic mechanical analysis (DMA) in 3-point bending mode. FT-IR was performed to ensure that there was no residual free NCO remaining Cell culture studies were conducted at Department of Biomedical Engineering at Carnegie Mellon University. Disc of approximately 250 microns were cut from a cylindrical composite sample and were sterilized. The discs were then placed in a well plate and MC3T3 cells were seeded on the composite surface in osteogenic media. Cell proliferation was verified by using Proliferating Nuclear Cell Antigen (PCNA) staining.

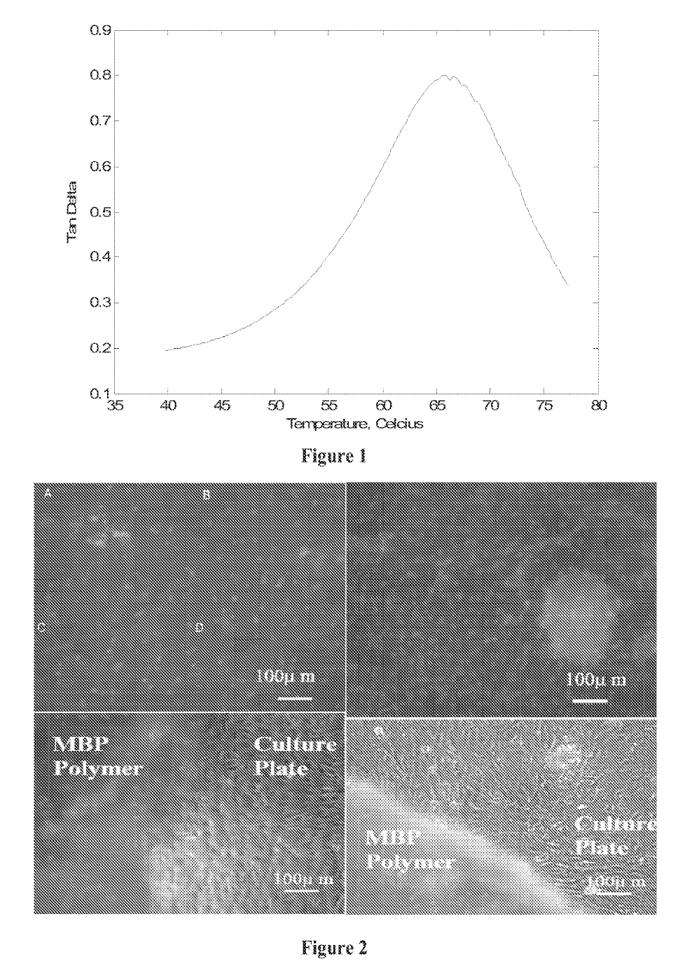

Results. A gel time of 20 minutes was achieved with 3420 ppm for Coscat 83 and 4760 ppm for Tegoamin 33. An exotherm of approximately 8° C. was observed during the hardening of the composite. The compression data shows that the composites synthesized with the 900 MW polyol has a modulus and stress at yield of 115.9 MPa and 21.1 MPa, respectively. An increase in both modulus and stress at yield was observed for the composites synthesized with the 300 MW polyol with values of 836.6 and 68.1, respectively. Initial results also show the modulus increased from 1098 MPa for non-etched MBP to 1160 MPa for etched MBP. The glass transition temperature, obtained from DMA via temperature sweeps, was observed to be 67° C., as shown FIG. 1. Preliminary results show that index has a large effect on mechanical properties, with a maximum in the modulus occurring at an index of 150 FT-IR spectra show that there is not residual free NCO, indicating that the reaction are fully completed. The results from in vitro studies (FIG. 2) have shown that the MBP/PEUR composites support cell attachment with cell viability greater than 90%.

Quasi-Prepolymer Synthesis: A quasi-prepolymer was synthesized using a modification of known procedures^{1,2,3}. To a dry 50 mL three neck round bottomed flask, 0.014 g of catalyst (Coscat 83) was added, followed by 17.639 g of lysine diisocyanate (LDI). The flask was equipped with argon purge, a magnetic stir bar, and a condenser. The mixture was heated to 90° C. using an oil bath. The condenser was attached to one neck, while the other two necks were plugged with rubber stoppers. Once the Cosact 83 and LDI mixture reached 90° C., 2.363 g of polycaprolactone (PCL 300) was added drop wise to the flask using a syringe over a period of approximately 5 minutes, while rapidly stirring. The reaction was allowed to occur for 3 hours under argon gas. The product was stored in a jar under argon and refrigerated. A viscous, semi-clear liquid was produced with a mass was 19.121 g, which correlates to a product yield was approximately 95%.

Poly(ester urethane)(PEUR)/Mineralized Bone Composite (MBP) Synthesis: The PEUR/MBP composites were synthesized using a reactive liquid molding process. The hardener was prepared by mixing the appropriate amounts of PCL 300, Coscat 83, and MBP (provided by Osteotech, Inc.) in a 10-mL cup at 3300 rpm fro 30 s in a Hauschild mixer. The quasi-prepolymer was then added and mixed with the hardener at 3300 rpm for 15 s. The resulting reactive liquid mixture was then cast into a cylindrical metal mold (provided by Osteotech, Inc.) equipped with two metal “plungers”. One plunger was placed at the bottom of the mold, while the other plunger was used to apply a large amount of hand pressure on the top to the reactive liquid mixture for a period of 1 minute. The mixture was allowed to stay in the mold for approximately 5 minutes. After the 5 minute gel time, the PEUR/MBP composite was then removed from the mold by removing the bottom plunger and pressing the top plunger by hand. The composites maintained shape integrity when they were de-molded, and they were then placed in an oven to cure at 60° C. for 18 hours. After the curing process, there were no changes in the dimensions of the cylinders; the cylinders were hard and tan, brown in color. Table 1, below, gives the composition of each cylinder that was synthesized.

TABLE 1
PEUR/MBP composite composition.

| Cylinder | Mass QP | Mass PCL300 | Mass catalyst | Mass MBP |
|---|---|---|---|---|
| I.D. | (g) | (g) | (g) | (g) |
| 1JD26-1 | 0.837 | 0.468 | 0.0054 | 3.166 |
| 1JD26-2 | 0.680 | 0.471 | 0.0057 | 3.168 |
| 1JD26-3 | 0.677 | 0.487 | 0.0076 | 3.126 |
| 1JD27-1 | 0.510 | 0.346 | 0.0060 | 3.406 |
| 1JD27-2 | 0.571 | 0.352 | 0.0061 | 3.413 |
| 1JD27-3 | 0.508 | 0.347 | 0.0060 | 3.401 |
| 1JD28-1 | 0.452 | 0.294 | 0.0068 | 3.539 |
| 1JD28-2 | 0.505 | 0.303 | 0.0073 | 3.599 |
| 1JD28-3 | 0.448 | 0.294 | 0.0070 | 3.689 |
| 1JD29-1 | 0.373 | 0.241 | 0.0063 | 3.763 |
| 1JD29-2 | 0.361 | 0.239 | 0.0087 | 3.761 |
| 1JD29-3 | 0.351 | 0.237 | 0.0085 | 3.746 |
| 1JD30-1 | 0.268 | 0.171 | 0.0071 | 3.890 |
| 1JD30-2 | 0.275 | 0.171 | 0.0099 | 3.904 |
| 1JD30-3 | 0.253 | 0.193 | 0.0071 | 3.176 |

The invention thus being described, it will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification, including the Example, be considered as exemplary only, and not intended to limit the scope and spirit of the invention.

Unless otherwise indicated, all numbers expressing quantities of ingredients, properties such as reaction conditions, and so forth used herein are to be understood as being modified in all instances by the term “about.” Accordingly, unless indicated to the contrary, the numerical parameters set forth in the herein are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this application, various publications are referenced. All such references, specifically including those listed below, are incorporated herein by reference.

### REFERENCES

Boyce T M, Winterbottom J M, Lee S, Kaes D R, Belaney R M,
Shimp L A, Knaack D. Cellular Penetration And Bone Formation Depends Upon Allograft Bone Fraction In A Loadbearing Composite Implant. 2005. p 133.

Guelcher S A, Patel V, Gallagher K, Connolly S, Didier J E, Doctor J, Hollinger J O. Synthesis and biocompatibility of polyurethane foam scaffolds from lysine diisocyanate and polyester polyols. Tissue Eng 2006; 12(5):1247-1259.
Guelcher S A, Gallagher K M, Srinivasan A, McBride S B, Didier J E, Doctor J S, Hollinger J O. Synthesis, in vitro biocompatibility and biodegradation, and mechanical properties of two-component polyurethane scaffolds: effects of water and polyol composition. Tissue Eng In Press.
Guelcher S A, Didier J E, Srinivasan A, Hollinger J O. Synthesis, mechanical properties, biocompatibility, and biodegradation of cast poly(ester urethane)s from lysine-based polyisocyanates and polyester triols. Manuscript in preparation.

We claim:

1. A bone composite material comprising lysine diisocyanate (LDI), polycaprolactone, and mineralized bone powder (MBP).

2. The material of claim 1, wherein the polycaprolactone is of a molecular weight of about 300 (PCL 300).

3. The material of claim 1, wherein the bone powder is about 100-500 microns.

4. The material of claim 1, wherein the bone is present in an amount of about 75 wt %.

5. The material of claim 1, wherein the bone is present in an amount of about 85 wt %.

6. The material of claim 1, wherein the bone is present in an amount of about 60 to about 83 wt %.

7. The material of claim 1, wherein the bone is present in an amount of about 70 to about 80 wt %.

8. The material of claim 1, being flowable.

9. The material of claim 1, wherein the bone powder is surface activated.

10. A composite comprising:
mineralized allograft bone; and
at least one degradable polyurethane that has a quasi-prepolymer and a resin mix, the resin mix having a polyester polyol and a catalyst; wherein the composite has a compression strength of greater than about 10 MPa and a modulus of greater than about 1 GPa.

11. The composite of claim 1, wherein the bone is powdered and present in a range of from about 1-90 wt %, or about 50-85 wt %, or about 70-85 wt %.

12. The composite of claim 1, being flowable.

13. A method of treating a wound, comprising:
providing mixture of mineralized allograft bone; and
at least one degradable polyurethane that has a quasi-prepolymer and a resin mix, the resin mix having a polyester polyol and a catalyst;
injecting the mixture into a wound site.

14. The method of claim 13, wherein the wound site is a bone.

15. A method of treating a wound, comprising:
providing mixture of mineralized allograft bone; and
at least one degradable polyurethane that has a quasi-prepolymer and a resin mix, the resin mix having a polyester polyol and a catalyst;
compression molding the mixture; and
contacting the molding to the wound site.

16. The method of claim 15, wherein the wound site is a bone.

17. A method for preparing biodegradable polyurethanes comprising: contacting a flowable quasi-prepolymer comprising free aliphatic polyisocyanate compounds with a polyester polyol hardener having a functionality of at least two and mineralized bone powder to form a reactive liquid mixture.

18. The method of claim 17, wherein the quasi-prepolymer is formed by contacting a polyisocyanate component comprising at least one aliphatic polyisocyanate compound with a polyol component comprising at least one polyol compound to form an adduct of the polyisocyanate component and the polyol component wherein a sufficient excess of the polyisocyanate component is used to form the quasi-prepolymer.

19. The method of claim 18 wherein the polyester polyol comprises poly(ethylene adipate), poly(ethylene glutarate), poly(ethylene azelate), poly(trimethylene glutarate), poly(pentamethylene glutarate), poly(diethylene glutarate), poly(diethylene adipate), poly(triethylene adipate), poly(1,2-propylene adipate), a mixture thereof, or a copolymer of at least two thereof.

20. The method of claim 15 wherein the polyester polyol comprises polyesters prepared from at least one of ε-caprolactone, glycolide or DL-lactide.

21. (canceled)

22. A bone scaffold comprising a biodegradable polyurethane mixture as set forth in claim 1.

23. A bone scaffold comprising a biodegradable polyurethane mixture as set forth in claim 10.

24. A bone scaffold comprising a biodegradable polyurethane mixture as set forth in claim 7.

25. A bone scaffold comprising a biodegradable polyurethane mixture as set forth in claim 11.
